(19) United States
(12) Patent Application Publication
(10) Pub. No.: US 2011/0130369 A1
(43) Pub. Date: Jun. 2, 2011

(54) USE OF FTS FOR THE TREATMENT OF MYOCARDIAL ISCHEMIA/REPERFUSION INJURY

(75) Inventors: Yoel Kloog, Herzliya (IL); Jakob George, Tel-Aviv (IL); Gad Keren, Kiryat Ono (IL); Rakefet Pando, Ramat-Gan (IL)

(73) Assignee: RAMOT AT TEL-AVIV UNIVERSITY LTD., Tel Aviv (IL)

(21) Appl. No.: 12/995,835

(22) PCT Filed: Jun. 4, 2009
(86) PCT No.: PCT/IL09/00563

§ 371 Date: Jan. 5, 2011

Related U.S. Application Data
(60) Provisional application No. 61/131,010, filed on Jun. 5, 2008.

(51) Int. Cl. A61K 31/60; A61P 9/10

(52) U.S. Cl. 514/159

(57) ABSTRACT

Disclosed are methods for reducing the extent of myocardial ischemia/reperfusion injury, comprising administering to a human prior to reperfusion of the ischemic myocardium or pre-angioplasty, coronary artery bypass surgery, or thrombolytic therapy an effective amount of FTS, or various analogs thereof, or a pharmaceutically acceptable salt thereof. Methods of treating ischemia/reperfusion injury, comprising administering to a human after reperfusion of the ischemic myocardium or post-angioplasty, coronary artery bypass surgery, or thrombolytic therapy an effective amount of FTS, or various analogs thereof, or a pharmaceutically acceptable salt thereof are also disclosed.

### CROSS-REFERENCE TO RELATED APPLICATIONS

This application claims the benefit of the filing date of U.S. Provisional Patent Application No. 61/131,010, filed Jun. 5, 2008, the disclosure of which is hereby incorporated herein by reference.

### BACKGROUND OF THE INVENTION

Myocardial ischemia associated with coronary artery disease is a leading cause of morbidity and mortality in the United States. [Tang Y L, et al., Hypertension 43:746-751 (2004)]. Myocardial ischemia is a condition characterized by reduced blood supply to the heart muscle, usually due to coronary artery disease (atherosclerosis of the coronary arteries). Severe interruption of the blood supply to the myocardial tissue may result in necrosis of cardiac muscle (myocardial infarction). Risk of myocardial ischemia increases with age, smoking, hypercholesterolemia (high cholesterol levels), diabetes, hypertension (high blood pressure) and is more common in men and those who have close relatives with ischaemic heart disease. Depending on the symptoms and risk, treatment of ischemia may be with medication, percutaneous coronary intervention (angioplasty) or coronary artery bypass surgery. [See Verma S, et al., Circulation 105:2332-2336 (2002)].

When a tissue becomes ischemic, a sequence of biochemical events is initiated that may lead to cellular dysfunction and necrosis. Resumption of coronary blood flow, otherwise known as reperfusion, is necessary to resuscitate the ischemic or hypoxic myocardium. Reperfusion of an ischemic area may result, however, in paradoxical cardiomyocyte dysfunction, a phenomenon termed “reperfusion-induced myocardial injury.” Thus, the net injury from a transient decrease or interruption of blood flow is the sum of two components—the direct injury occurring during the ischemic interval and the indirect or reperfusion injury that follows. When there is a long duration of ischemia, the “direct” damage resulting from hypoxia alone is the predominant mechanism. The shorter the duration of ischemia, the more important indirect or reperfusion-induced damage becomes. For the cardiologist, ischemia/reperfusion injury (“I/R injury”) occurs following successful angioplasty (“angioplasty-induced cardiac ischemia”), drug-induced thrombolysis, and coronary artery bypass surgery. [See Id.].

Myocardial injury that has developed through a period of ischemia/reperfusion may have many causes. Past research concentrated on the mechanisms causing cellular injury during ischemia and on protective procedures designed to reduce development of ischemic injury. [Piper H M, Meuter K, Schafer C, Ann Thorac Surg 75:S644-8 (2003)]. As discussed above, the readmission of oxygenated blood into previously ischemic myocardium can initiate a cascade of events that will paradoxically produce additional myocardial cell dysfunction and cell death. [Lefer D J, Granger D N, Am J Med 109:315-23 (2000)]. The cellular mechanisms involved in the pathogenesis of myocardial I/R injury are complex and are believed to involve the interaction of a number of cell types, including coronary endothelial cells, circulating blood cells, and cardiac myocytes [Lucchesi B R, Annu Rev Physiol 52:561-576 (1990); Lefer D J, Nakanishi K, Vinten-Johansen J, Ma X L, Lefer A M, Am J Physiol 263:H850-H1246 (1992); Van Benthuysen K M, McMurtry I F, Horwitz L D, J Clin Invest 79:265-274 (1987)], most of which are capable of generating reactive oxygen species (ROS). These ROS play an important role in the progression and aggravation of heart failure, and can induce contractile dysfunction and myocardial structural damage. [Hochhauser E, Kaminski O, Shalom H, Leshem D, Shneyvays V, Shainberg A, Vidne B A, Antioxid Redox Signal 6(2):335-44 (2004)].

Apoptosis is an active gene-directed cell death process which plays a key role in myocardial reperfusion injury. [Aoki H, Kang P M, Hampe J, Yoshimura K, Noma T, Matsuzaki M, Izumo S, J. Biol. Chem. 277:10244-10250 (2002)]. Cardiac myocyte cell death triggered by ischemia/reperfusion can occur by both apoptosis and necrosis. While cell death after prolonged periods of ischemia is ascribed to necrosis, apoptosis occurs in cells and tissues exposed to reoxygenation after ischemia. [Ferrandi C, Ballerio R, Gaillard P, Giachetti C, Carboni S, Vitte P A, Gotteland J P, Cirillo R, Br J Pharmacol 142(6):953-60 (2004)]. The intracellular signaling pathways that mediate stress responses of cardiomyocytes remain not fully delineated.

### SUMMARY OF THE INVENTION

A first aspect of the present invention is directed to a method of reducing the extent of myocardial ischemia/reperfusion injury. The method comprises administering to a human prior to reperfusion of the ischemic myocardium an effective amount of S-farnesylthiosalicylic acid (FTS) or an analog thereof, or a pharmaceutically acceptable salt thereof.

Another aspect of the present invention is directed to a method of reducing the extent of myocardial ischemia/reperfusion injury. The method comprises administering to a human prior to angioplasty, coronary artery bypass surgery, or thrombolytic therapy, such as with tissue plasminogen activator (tPA) or streptokinase (SK), an effective amount of S-farnesylthiosalicylic acid (FTS) or an analog thereof, or a pharmaceutically acceptable salt thereof.

Another aspect of the present invention is directed to a method of treating myocardial ischemia/reperfusion injury. The method comprises administering to a human after reperfusion of the ischemic myocardium an effective amount of S-farnesylthiosalicylic acid (FTS) or an analog thereof, or a pharmaceutically acceptable salt thereof.

Another aspect of the present invention is directed to a method of treating myocardial ischemia/reperfusion injury. The method comprises administering to a human post-angioplasty, coronary artery bypass surgery, or thrombolytic therapy, such as with tissue plasminogen activator (tPA) or streptokinase (SK), an effective amount of S-farnesylthiosalicylic acid (FTS) or an analog thereof, or a pharmaceutically acceptable salt thereof.

The compound is typically administered in the form of a composition, which is formulated with at least one pharmaceutically acceptable inert ingredient (e.g., a carrier, vehicle, etc.). Modes of administration include oral and intravenous protocols.

### BRIEF DESCRIPTION OF THE DRAWINGS

FIG. 1A is a bar graph illustrating the effect of FTS heart perfusion on the hemodynamic measurement of coronary flow (CF) [ml/min] taken at stabilization period and every 10 minutes during the reperfusion period as compared to control ex vivo. The measurements are presented as a percentage of the baseline data shown in Table 1. R=reperfusion (min). Columns, mean bars ±SE.

FIG. 1B is a bar graph illustrating the effect of FTS heart perfusion on the hemodynamic measurement of left ventricular pressure (LVP) [mmHg] taken at stabilization period and every 10 minutes during the reperfusion period as compared to control ex vivo. The measurements are presented as a percentage of the baseline data shown in Table 1. R=reperfusion (min). Columns, mean bars ±SE. *p<0.05 compared to control.

FIG. 1C is a bar graph illustrating the effect of FTS heart perfusion on the hemodynamic measurement of positive dP/dt [mmHg/sec] taken at stabilization period and every 10 minutes during the reperfusion period as compared to control ex vivo. The measurements are presented as a percentage of the baseline data shown in Table 1. R=reperfusion (min). Columns, mean bars ±SE. *p<0.05 compared to control.

FIG. 1D is a bar graph illustrating the effect of FTS heart perfusion on the hemodynamic measurement of negative dP/dt (ndP/dt) [mmHg/sec] taken at stabilization period and every 10 minutes during the reperfusion period as compared to control ex vivo. The measurements are presented as a percentage of the baseline data shown in Table 1. R=reperfusion (min). Columns, mean bars ±SE. *p<0.05 compared to control.

FIG. 1E is a bar graph illustrating the effect of FTS heart perfusion on the hemodynamic measurement of heart rate (HR) [beats/min] taken at stabilization period and every minutes during the reperfusion period as compared to control ex vivo. The measurements are presented as a percentage of the baseline data shown in Table 1. R=reperfusion (min). Columns, mean bars ±SE.

FIG. 2A is a bar graph illustrating the release of creatine phosphokinase (CK) [U/min] to the coronary effluent in FTS-perfused hearts taken at stabilization period and at various time points during the reperfusion period (1, 10, and 30 min) as compared to control ex vivo. Columns, mean bars ±SE. *p<0.05 compared to control.

FIG. 2B is a bar graph illustrating the release of lactate dehydrogenase (LDH) [U/min] to the coronary effluent in FTS-perfused hearts taken at stabilization period and at various time points during the reperfusion period (1, 10, and 30 min) as compared to control ex vivo. Columns, mean bars ±SE. *p<0.05 compared to control.

FIG. 3A are photographic images depicting ischemia/reperfusion damage by TTC staining of an FTS-perfused heart (right) as compared to control (left) revealing the reduced regions of irreversible ischemic injury in an FTS-perfused heart (right) as compared to control (left) ex vivo.

FIG. 3B is a bar graph illustrating the decreased percentage of irreversible damage in the heart tissue after ischemia and FTS perfusion as compared to control as determined by scanning the images of heart ventricular sections stained with TTC and determining the percentage of irreversible injury from the total area of the section. Columns, mean bars ±SE. *p<0.05 compared to control.

FIG. 4A are Western immunoblot images illustrating the effect of FTS on Ras and its downstream effectors in both FTS-perfused hearts and control ex vivo.

FIG. 4B is a bar graph illustrating the results of the quantitative analysis of the immunoblots indicating a significant reduction in Ras (Ras-GTP) and p-Jnk in hearts of the FTS group (*p<0.05) and no significant differences in total Ras, Jnk, P38, p-P38, Mst1 and p-Mst1 levels between both groups ex vivo.

FIG. 5A is a bar graph illustrating the results of the echocardiographic studies revealing that infarcted rats administered FTS (5 mg/kg) according to Protocol A (prophylactic regimen) demonstrated significantly improved left-ventricular end-diastolic area (LVEDA) than compared to the control I/R in vivo. Columns, mean bars ±SE. *p<0.05 compared to control I/R.

FIG. 5B is a bar graph illustrating the results of the echocardiographic studies revealing that infarcted rats administered FTS (5 mg/kg) according to Protocol A (prophylactic regimen) demonstrated significantly improved left-ventricular end-systolic area (LVESA) than compared to the control I/R in vivo. Columns, mean bars ±SE. *p<0.05 compared to control I/R.

FIG. 5C is a bar graph illustrating the results of the echocardiographic studies revealing that infarcted rats administered FTS (5 mg/kg) according to Protocol A (prophylactic regimen) demonstrated significantly higher fractional shortening (FS) of area (%) compared to control I/R rats in vivo. Columns, mean bars ±SE. *p<0.05 compared to control I/R.

FIG. 5D is a bar graph illustrating the results of the echocardiographic studies revealing that infarcted rats administered FTS (5 mg/kg) according to Protocol A (prophylactic regimen) demonstrated significantly greater septum diameter (cm) compared to control I/R rats in vivo. Columns, mean bars ±SE. *p<0.05 compared to control I/R.

FIG. 6A is a bar graph illustrating the histological evaluation of irreversible damage area revealing that infarcted rats administered FTS (5 mg/kg) according to Protocol A (prophylactic regimen) demonstrated significantly reduced scar area of irreversible damage (% of left ventricular wall) compared to control I/R rats in vivo. Columns, mean bars ±SE. *p<0.05 compared to control I/R.

FIG. 6B are a series of microscopic images of typical stained sections (H&E and Masson Trichrome) of the hearts from all in vivo experimental and control groups used to evaluate cardiomyocyte morphology and quantify collagen deposition.

FIG. 7A are Western immunoblot images illustrating the beneficial effect of FTS (5 mg/kg) administered according to Protocol A (prophylactic regimen) on Ras signaling in the in vivo model of I/R as compared to control.

FIG. 7B is a bar graph illustrating the results of the quantitative analysis of the immunoblots demonstrating a significant reduction in levels of total Ras, active Ras (Ras-GTP), and p-Mst1 and no difference in levels of Mst, P38, p-P38, Jnk, and p-Jnk in both groups administered FTS according to Protocol A in vivo. Columns, mean bars ±SE. *p<0.05 compared to control I/R.

### DETAILED DESCRIPTION

FTS and its analogs useful in the present invention are represented by formula I:

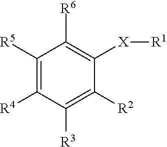

wherein
R¹ represents farnesyl or geranylgeranyl;
R² is COOR⁷, or CONR⁷R⁸, wherein R⁷ and R⁸ are each independently hydrogen, alkyl or alkenyl;
R³, R⁴, R⁵ and R⁶ are each independently hydrogen, alkyl, alkenyl, alkoxy, halo, trifluoromethyl, trifluoromethoxy, or alkylmercapto; and
X represents S.

The structure of FTS is as follows:

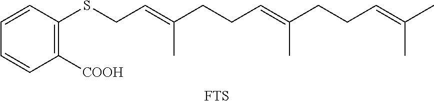

FTS analogs embraced by formula I, and which may be suitable for use in the present invention, include 5-fluoro-FTS, 5-chloro-FTS, 4-chloro-FTS, S-farnesyl-thiosalicylic acid methyl ester (FTSME), and S-geranylgeranyl-thiosalicylic acid (GGTS). Structures of these compounds are set forth below.

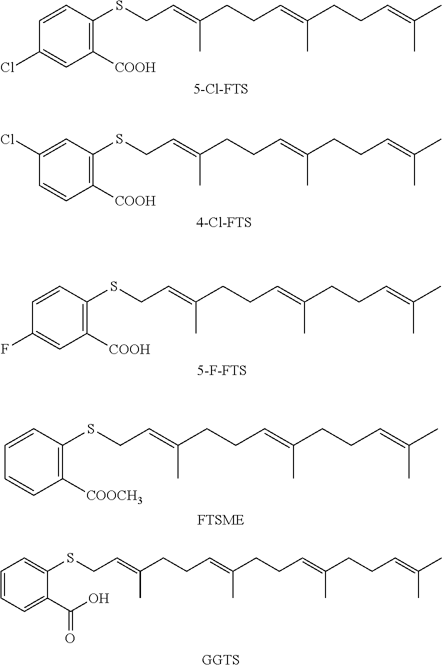

Amides of FTS embraced by formula I may be suitable for use in the present invention, e.g., FTS-amide, wherein R¹=farnesyl; R²=CONR⁷R⁸ where R⁷ and R⁸ are each H; R³, R⁴, R⁵, and R⁶ are each H, and X=S.

In some embodiments, GGTS, 5-fluoro-FTS, FTSME, or FTS-amide may be administered in accordance with the present invention.

Methods for preparing the compounds of formula I are disclosed in U.S. Pat. Nos. 5,705,528 and 6,462,086. See also, Marom M, Haklai R, Ben-Baruch G, Marciano D, Egozi Y, Kloog Y, J Biol Chem 270:22263-70 (1995).

Pharmaceutically acceptable salts of the Ras antagonists of formula I may be useful. These salts include, for example, sodium and potassium salts. Other pharmaceutically acceptable salts may be selected in accordance with standard techniques as described in Berge S M, Bighley L D, and Monkhouse D C, J. of Pharm. Sci. 66(1):1-19 (1977). In preferred embodiments, however, FTS and its analogs are not administered in the form of a salt (i.e., they are administered in non-salified form).

As used herein, the term “effective amount” refers to the dosage(s) of FTS or its analogs or salts that is effective for prophylaxis and for the treating, and thus includes dosage amounts that inhibit or reduce the likelihood of onset of reperfusion injury, or ameliorate existing ischemia/reperfusion injury and its associated manifestations, diminish duration or extent of injury, delay or slow injury progression, or prolong patient survival. The average daily dose of FTS generally ranges from about 50 mg to about 2000 mg, and in some embodiments, from about 200 mg to about 1600 mg. According to Phase I human clinical trials (for various cancers) conducted by Concordia Pharmaceuticals, Inc., S-farnesylthiosalicylic acid (FTS, salirasib) is a well-tolerated compound without dose-limiting toxicities at doses up to 1600 mg/day.

The frequency of administration, dosage amounts, and the duration of treatment using FTS prior to, substantially simultaneously with, or after reperfusion of the ischemic myocardium may depend on several factors, e.g., the overall health, size and weight of the patient, the patient's tolerance to the drug, and the particular regimen being administered. For example, duration of administering FTS may last seconds, minutes, an hour, a day, a week, a year, a predetermined interval sufficient to inhibit or reduce the likelihood of onset of reperfusion injury, or ameliorate existing I/R injury and its associated manifestations, diminish duration or extent of I/R injury, delay or slow I/R injury progression, or prolong patient survival, or until recovery from I/R injury.

In some embodiments, wherein the use is prophylactic, FTS is administered prior to surgery or prior to angioplasty, coronary bypass surgery, or thrombolytic therapy and the resulting reperfusion of the ischemic myocardium. In these embodiments, the dosing scheme is designed to produce and maintain an effective amount of FTS in the bloodstream of the patient during the reperfusion event. Thus, the amounts of FTS, the frequency of administration, and the duration of treatment can be adjusted accordingly. FTS may be administered orally at any time prior to reperfusion of the ischemic myocardium, e.g., minutes, days, or weeks, depending, for example, on the severity of the ischemic event and the timing of the reperfusion. For example, FTS can be administered on a daily basis, e.g., each in single once-a-day or divided doses. Thus, FTS may be administered prior to treatment, e.g., prior to reperfusion of the ischemic myocardium, prior to angioplasty, coronary artery bypass surgery, or thrombolytic therapy and continuing daily for a predetermined period. In these embodiments, for example, the average daily dose of FTS generally ranges from about 25 mg to about 1000 mg, and in some embodiments, from about 50 mg to about 400 mg.

In other embodiments, wherein the use is directed to treatment of an existing I/R injury, FTS is administered after surgery or post-reperfusion. In these embodiments, the dosing scheme is designed to produce and maintain an effective amount of FTS in the bloodstream of the patient after the reperfusion event. Thus, the amounts of FTS, the frequency of administration, and the duration of treatment can be adjusted accordingly. FTS may be administered at any time following reperfusion of the ischemic myocardium, e.g., seconds, minutes, days, or weeks. For example, FTS can be administered on a daily basis, e.g., each in single once-a-day or divided doses. Thus, FTS may be administered after surgery or reperfusion of the ischemic myocardium or post-angioplasty, coronary artery bypass surgery, or thrombolytic therapy and continuing daily for a predetermined period. In these embodiments, the average daily dose of FTS generally ranges from about 200 mg to about 2000 mg, and in some embodiments, from about 400 mg to about 1600 mg.

In another embodiment, FTS is administered substantially simultaneously with surgery or reperfusion of the ischemic myocardium, substantially simultaneously to angioplasty, coronary artery bypass surgery, or thrombolytic therapy and continuing daily for a predetermined period. Thus, FTS is administered concurrently with surgery or during reperfusion of the ischemic myocardium. In these embodiments, the dosing scheme is designed to produce and maintain an effective amount of FTS in the bloodstream of the patient substantially simultaneously with reperfusion. Thus, FTS may be administered in a single intravenous dose concurrent with the reperfusion procedure and may continue with daily oral treatment for a predetermined period. In these embodiments, the average single intravenous dose of FTS generally ranges from about 25 mg to about 600 mg, and in some embodiments, from about 25 mg to about 200 mg, and the average daily oral dose of FTS generally ranges from about 25 mg to about 1000 mg, and in some embodiments from about 50 mg to 400 mg.

The methods of the present invention may be used for prophylaxis of I/R injury. In other embodiments, the methods of the present invention may be used for treatment of existing I/R injury. In preferred embodiments, FTS is administered orally. In an oral dosage form, the FTS is typically present in a range of about 25 mg to about 500 mg, and in some embodiments, from about 25 mg to about 200 mg.

In some embodiments, FTS may be administered by dosing orally on a daily basis for 3 or 4 weeks (e.g., beginning prior to or after surgery resulting in reperfusion of the ischemic myocardium), followed by a one-week “off period”, and repeating for a predetermined interval sufficient to inhibit or reduce the likelihood or onset of reperfusion injury, or ameliorate existing I/R injury and its associated manifestations, diminish duration or extent of I/R injury, delay or slow I/R injury progression, or prolong patient survival, or until recovery from I/R injury. In another embodiment, FTS may be administered either before or after surgery by dosing twice daily without an “off period” and until recovery from I/R injury. Parenteral administration may also be suitable.

In another embodiment, the dosing regimen may entail administration with oral FTS (e.g., a capsule or a tablet) continuously without interruption (i.e., without an “off period” of one or more days). FTS is not required to be delivered in any specific manner, and may be delivered in conjunction with other therapies. Thus, dosing regimens for administering FTS may be adjusted to meet the particular needs of the patient.

Oral compositions for FTS and its analogs and salts (the active agent) for use in the present invention can be prepared by bringing the active agent(s) into association with (e.g., mixing with) a pharmaceutically acceptable carrier or vehicle (e.g., a pharmaceutically acceptable inert ingredient). Suitable carriers are selected based in part on the mode of administration. Carriers are generally solid or liquid in nature. In some cases, compositions may contain both solid and liquid carriers. Compositions suitable for oral administration that contain the active agent are preferably in solid dosage forms such as tablets (e.g., including film-coated, sugar-coated, controlled or sustained release), capsules, e.g., hard gelatin capsules (including controlled or sustained release) and soft gelatin capsules, powders and granules. The compositions, however, may be contained in other carriers that enable administration to a patient in other oral forms, e.g., a liquid or gel. Regardless of the form, the composition is divided into individual or combined doses containing predetermined quantities of the active agent.

Oral dosage forms may be prepared by mixing the active pharmaceutical ingredient or ingredients with one or more appropriate carriers (optionally with one or more other pharmaceutically acceptable additives or excipients), and then formulating the composition into the desired dosage form, e.g., compressing the composition into a tablet or filling the composition into a capsule or a pouch. Typical carriers and excipients include bulking agents or diluents, binders, buffers or pH adjusting agents, disintegrants (including crosslinked and super disintegrants such as croscarmellose), glidants, and/or lubricants, including lactose, starch, mannitol, microcrystalline cellulose, ethylcellulose, sodium carboxymethylcellulose, hydroxypropylmethylcellulose, dibasic calcium phosphate, acacia, gelatin, stearic acid, magnesium stearate, corn oil, vegetable oils, and polyethylene glycols. Coating agents such as sugar, shellac, and synthetic polymers may be employed, as well as colorants and preservatives. See, Remington's Pharmaceutical Sciences, The Science and Practice of Pharmacy, 20th Edition, (2000).

Liquid form compositions include, for example, solutions, suspensions, emulsions, syrups, and elixirs. The active agent, for example, can be dissolved or suspended in a pharmaceutically acceptable liquid carrier such as water, an organic solvent (and mixtures thereof), and/or pharmaceutically acceptable oils or fats. Examples of liquid carriers for oral administration include water (particularly containing additives as above, e.g., cellulose derivatives, preferably in suspension in sodium carboxymethyl cellulose solution), alcohols (including monohydric alcohols (including monohydric alcohols and polyhydric alcohols, e.g., glycerin and non-toxic glycols) and their derivatives, and oils (e.g., fractionated coconut oil and arachis oil). The liquid composition can contain other suitable pharmaceutical additives such as solubilizers, emulsifiers, buffers, preservatives, sweeteners, flavoring agents, suspending agents, thickening agents, colorants, viscosity regulators, stabilizers or osmoregulators.

Carriers suitable for preparation of compositions for parenteral administration include Sterile Water for Injection, Bacteriostatic Water for Injection, Sodium Chloride Injection (0.45%, 0.9%), Dextrose Injection (2.5%, 5%, 10%), Lactated Ringer's Injection, and the like. Dispersions can also be prepared in glycerol, liquid polyethylene glycols and mixtures thereof, and in oils. Compositions may also contain tonicity agents (e.g., sodium chloride and mannitol), antioxidants (e.g., sodium bisulfite, sodium metabisulfite and ascorbic acid) and preservatives (e.g., benzyl alcohol, methyl paraben, propyl paraben and combinations of methyl and propyl parabens).

In order to fully illustrate the present invention and advantages thereof, the following specific examples/experiments are given, it being understood that the same is intended only as illustrative and in no way limitative.

### Example 1

### Experimental Design

The purpose of these in vivo and ex vivo experiments was to assess the ability of FTS to reduce I/R injury and, thereby, its ability to improve cardiac function. Here the effects of FTS using both an isolated rat heart ex vivo and an in vivo I/R rodent model were examined. In other experiments, the perfused hearts were examined using digital photography, microscopy, and staining techniques to visualize and quantify viable and necrotic tissue, cardiomyocyte morphology, and collagen deposition. In another experiment, homogenates of the perfused hearts from both in vivo and ex vivo experimental models were examined using quantitative analysis of Western immunoblots. Additionally, hemodynamic measurements and biochemical measurements on the ex vivo models were analyzed. The primary goal of the experiments was to determine: (I) the effect of FTS perfusion on heart recovery ex vivo; (II) the effect of FTS on enzymatic leakage of CK and LDH into the coronary effluent ex vivo; (III) the effect of FTS on reducing regions of irreversible ischemic injury compared with control ex vivo; (IV) the effect of FTS on Ras signaling in perfused hearts ex vivo; (V) the effect of systemic administration of FTS on the heart in vivo; (VI) the effect of FTS on cardiomyocyte morphology and collagen deposition in vivo; and (VII) the effect of FTS on Ras signaling in vivo.

Overall, the results of the experiments described herein indicated that prophylactic administration of FTS, i.e., prior to reperfusion of the ischemic myocardium (e.g., a prophylactic regimen) improved cardiac function and lessens the extent of irreversible damage caused by I/R injury in vivo and ex vivo. In addition, the results provided herein provide a biochemical rationale for administration of FTS after reperfusion of the ischemic myocardium, (e.g., a treatment regimen). In vivo, post-ischemic treatment regimens using FTS at low doses (5 mg/kg), showed no significant difference from controls. Thus, in these embodiments, relatively higher doses of FTS are necessary, e.g., from about 10-100 mg/kg.

### Materials and Methods

### Animals

Male Lewis rats were purchased from Harlan and maintained in a local vivarium under conventional conditions.

### FTS Preparation for Isolated Heart Perfusion

FTS was provided by Concordia Pharmaceuticals, Inc. (Ft. Lauderdale, Fla.). FTS was stored in chloroform, which was evaporated under a stream of nitrogen immediately before use. The powder was dissolved in DMSO and diluted with Krebs-Henseleit bicarbonate buffer solution (KHB) to yield a 1 μM drug solution containing 10% DMSO.

### Isolated Heart Perfusion

Rats were heparinized (500 U/kg) and anesthetized (i.p.) with ether. The hearts were quickly removed, the aorta was cannulated and the heart perfused in retrograde according to Langendorff at a pressure of 96 cmH₂O with oxygenated Krebs-Henseleit bicarbonate buffer solution (KHB) containing (mM): 118 NaCl, 2.4 KCl, 1.2 MgSO₄, 7×H₂O, 2.5 CaCl₂, 5 EDTA, 1.2 KH₂PO₄, 25 NaHCO₃, 4 glucose at 37° C. [Hochhauser E, Kivity S, Offen D, Maulik N, Otani H, Barhum Y, et al., Am J Physiol Heart Circ Physiol 284:H2351-59 (2003)]. The isolated heart was stabilized for 20 minutes at a constant perfusion pressure and then subjected to 30 minutes of ischemia followed by 30 minutes of reperfusion. Ischemia was created by clamping the aortic cannula. During reperfusion, hearts were given KHB (buffer) with FTS or KHB only. The temperature of the heart (measured in the right ventricle) was maintained at 37°±0.2° C. throughout the experiment by a micro-thermocouple connected to a digital thermometer (Webster Laboratories Altadena, Calif.). In all stages of the protocol, the left ventricular developed pressure (LVP), the rate of pressure development and relaxation (±dP/dt) and the heart rate (HR), were continuously recorded by the CODAS data acquisition system (San Diego, Calif.). Rate pressure product (RPP), an index of myocardial workload, was calculated by multiplying LVP by HR. Coronary effluent was collected at one-minute intervals before and after ischemia, at various time points (1, 10 and 30 minutes reperfusion) and analyzed for CK activity (Boehringer Mannheim).

### Measurement of Irreversible Ischemic Injury

After 30 minutes of reperfusion, hearts were weighed and cut into sections. Middle sections were incubated with 2,3,5-triphenyl tetrazolium chloride (TTC) in phosphate buffer at 37° C. for 30 minutes. TTC stained the viable tissue red while the necrotic tissue remained discolored. Sections were fixed overnight in 2% paraformaldehyde. The sections were then placed between two cover slips and digitally photographed using a Fugi Finepixs1pro camera, with a resolution of 1400×960 pixels and quantified with IMAGE J 5.1 software. The area of irreversible injury (TTC-negative) is presented as a percentage of the entire area of the section [Maulik N, Yoshida T, and Das D K, Molecular and Cellular Biochemistry 196:13-21 (1999)].

### Left Anterior Descending (LAD) Artery Ligation

At the age of 12 weeks, rats were anesthetized (mixture of 8 mg/100 g ketamine, 5 mg/100 g xylazine), intubated, and ventilated with a Harvard Rodent Ventilator Model 383 (respiratory rate: 50/min, respiratory volume: 2.5 mL). A rectal thermocouple was used to continuously monitor body temperature, which was maintained at 37° C. using a heating pad. A left thoracotomy in the third intercostal space was performed to expose the heart. The location of the left descending coronary artery was identified and then occluded with a 6-0 silk suture. Occlusion was confirmed by monitoring the pallor of the region at risk, and an electrocardiogram was used to observe changes such as widening of QRS and ST-T segment elevation. After 30 minutes, the occlusion was removed, the thorax was closed, and rats were returned to their cages at the local vivarium. No death occurred in response to LAD occlusion, or drug injection.

### FTS Preparation for Injections

FTS was stored in chloroform, which was evaporated under a stream of nitrogen immediately before use. The powder was dissolved in absolute ethanol and diluted to the desired concentration in sterile PBS made basic with NaOH. Carrier solution (1000 μl) containing 1.35 mg of FTS (5 mg/kg) was injected intraperitoneally (i.p.) into each rat. Control solution was prepared at the same time starting with PBS and absolute ethanol.

### FTS Injections

LAD ligation was performed in two groups of rats. Group 1 received 5 mg/kg FTS and group 2 received PBS according to two protocols: (A) FTS or PBS was administered 7 days on a daily basis before LAD ligation, locally during occlusion opening (0.5 μM/100 μl), and continued for 14 days, every other day [prophylactic regimen] (B) FTS or PBS was administered 14 days after LAD ligation every other day [treatment regimen]. At day 14, rats were taken for echocardiography as previously described [Yitzhaki S, Shainberg A, Cheporko Y, Vidne B A, Sagie A, Jacobson K A, et al., Biochem Pharmacol 72(8):949-55 (2006)]. The rats were sacrificed on day 15. Hearts were taken for histological and immunological analyses.

### Histopathology

Hearts were removed and fixed in 4% formalin, then embedded in paraffin. Several transverse sections were cut from the paraffin-embedded samples and stained with hematoxylin and eosin. Sections from each heart were also stained with Masson trichrome. Slides were then assessed in a blinded fashion by a pathologist using light microscopy and scored for the percentage of LVP involvement and collagen deposition. Scar area was evaluated using data from both H&E and Masson staining.

Determination of Ras, Erk, p-Erk, Jnk, p-Jnk, P38, p-P38, Mst1, and p-Mst1

Hearts were obtained from FTS- and PBS-treated rats (LAD ligation experiments) and from isolated heart perfusion experiments. The hearts were homogenized in cold homogenization buffer containing protease inhibitors. Protein concentration was determined by the Bradford assay and samples containing 100 μg protein were used for determination of protein levels by Western immunoblotting using: pan-anti-Ras Ab (Ab03; Santa Cruz, Calif.), anti JNK Ab (Cell Signaling; Danvers, Mass.), anti p-JNK Ab (Cell Signaling), anti P38 Ab (Cell Signaling), anti p-P38 Ab (Cell Signaling), anti Mst1 Ab (Cell Signaling), and anti p-Mst1 Ab (Cell Signaling). Enhanced chemiluminescence (ECL) and densitometric analysis were performed as detailed previously [Haklai R, Weisz M G, Elad G, Paz A, Marciano D, Egozi Y, et al., Biochemistry 37(5):1306-14 (1998)1.

### Determination of Ras-GTP

The hearts were prepared as described above. Samples containing 0.5 mg protein used for determination of levels of active GTP-bound Ras by the glutathione S-transferase-RBD pull-down assay followed by Western immunoblotting with pan-anti-Ras Ab as detailed previously [Jansen B, Schlagbauer-Wadl H, Kahr H, Heere-Ress E, Mayer B X, Eichler H, et al., Proc Natl Acad Sci U.S.A. 96(24):14019-24 (1999)].

### Statistical Analysis

Results are expressed as means±standard error of the mean (SE). Values during stabilization period were defined as 100%. A statistical difference between the groups was assessed by analysis of variance (ANOVA) with repeated measurements using the multiple comparison option of Duncan. If differences were established, values were compared using Student's t-test: p<0.05 was considered significant.

### Results

### I. FTS Perfusion to the Isolated Ischemic Heart Improved Cardiac Recovery Ex Vivo.

To determine the protective effects of Ras protein inhibition on cardiac damage derived from I/R injury ex vivo, we first determined the baseline absolute values of the rats and cardiac function prior to ischemia in FTS (1 μM) and control groups. Table 1 (below) summarizes the baseline values of cardiac function and coronary flow in Langendorf-perfused isovolumically contracting rat hearts prior to ischemia revealing no significant differences in any of the measurements between FTS (1 μM) and control groups. Values are expressed as mean±SE.

TABLE 1

| | Control | FTS | |
|---|---|---|---|
| | n = 7 | n = 6 | P value |
| Total weight | 359.83 ± 25.53 | 348.5 ± 38.57 | 0.26 |
| (g) | | | |
| Heart weight | 1.57 ± 0.17 | 1.53 ± 0.17 | 0.35 |
| (g) | | | |
| CF total | 15 ± 2 | 14 ± 4 | 0.41 |
| (ml/min) | | | |
| CF/Heart | 9.2 ± 2.45 | 9.3 ± 2.95 | 0.46 |
| weight | | | |
| LVP (mmHg) | 224.3 ± 84.6 | 200 ± 19.4 | 0.25 |
| dP/dt | 4544.8 ± 1284.2 | 4126.3 ± 474.9 | 0.22 |
| (mmHg/sec) | | | |
| ndP/dt | 3832 ± 1177.2 | 3640.6 ± 587.7 | 0.35 |
| (mmHg/sec) | | | |
| HR (b/min) | 262.6 ± 43.4 | 265.3 ± 36.5 | 0.45 |
| RRP | 51,645 ± 14570 | 53,580 ± 12170 | 0.4 |
| (b/min*mmHg) | | | |

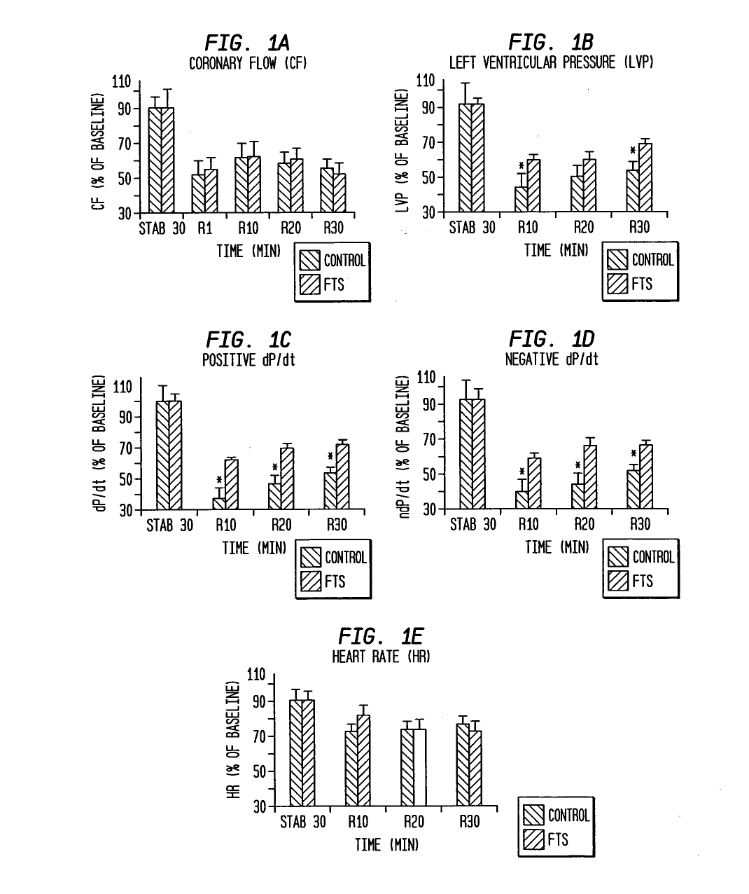

Next, we perfused the isolated rat hearts with FTS (1 μM) in the Langendorff system after 30 minutes of ischemia (FIG. 1). FTS had no effect on the CF recovery in the FTS vs. control group (FIG. 1A) during the 30 minutes of reperfusion. LVP and ±dP/dt recovery was significantly better in the FTS-perfused hearts compared to controls (p<0.05) (FIGS. 1B-1D). There was no significant difference in the recovery of HR between both groups (FIG. 1E). Thus, hearts that were perfused with FTS following ischemia showed better recovery in most hemodynamic parameters, as seen by left ventricular development pressure and the rate of pressure development and relaxation.

### II. FTS Perfusion to the Isolated Ischemic Heart Showed a Trend of Lower Enzymatic Leakage Ex Vivo.

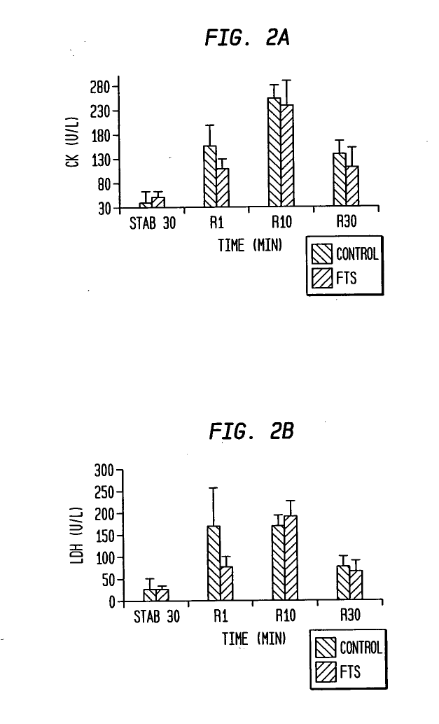

Next, to determine the effects of enzymatic leakage in coronary effluent, measurements of creatine phosphokinase (CK) and lactate dehydrogenase (LDH) concentration were conducted. Both CK and LDH concentration in effluent increased in both groups compared with the baseline measurements. At 1 minute and 30 minutes of reperfusion, CK and LDH release to the coronary effluent was found to be lower in the FTS group compared with control group, but did not reach statistical significance (FIGS. 2A and 2B).

### III. FTS Reduced Irreversible Ischemic Injury Ex Vivo.

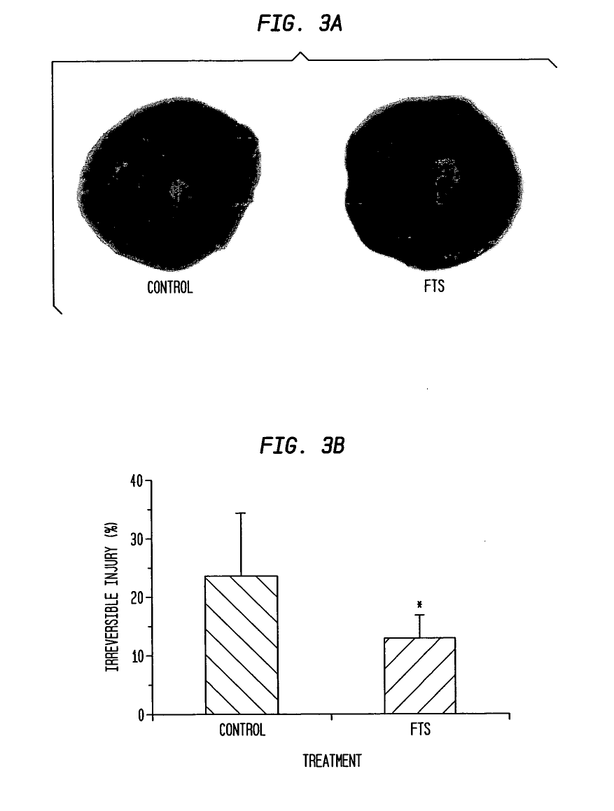

To evaluate the effect of FTS on reducing the severity of irreversible damage after ischemic injury, tetrazolium chloride (TTC) staining on the isolated heart models was performed. TTC staining revealed that FTS perfusion to the isolated heart subjected to 30 minutes ischemia and 30 minutes of reperfusion was accompanied with reduced regions of irreversible ischemic injury compared with the control group, (12.7±2% vs. 23.7±4% respectively, p<0.05) [FIG. 3B]. Typical sections showing viable and necrotic areas are shown (FIG. 3A). Thus, pre-treatment with FTS ex vivo reduced regions of irreversible ischemic injury in FTS-perfused hearts as compared to control.

IV. FTS Caused a Significant Decrease in the Downstream Effectors Ras-GTP and p-Jnk, While Total Ras, Jnk, P38, p-P38, Mst1 and p-Mst1 were not Affected Ex Vivo.

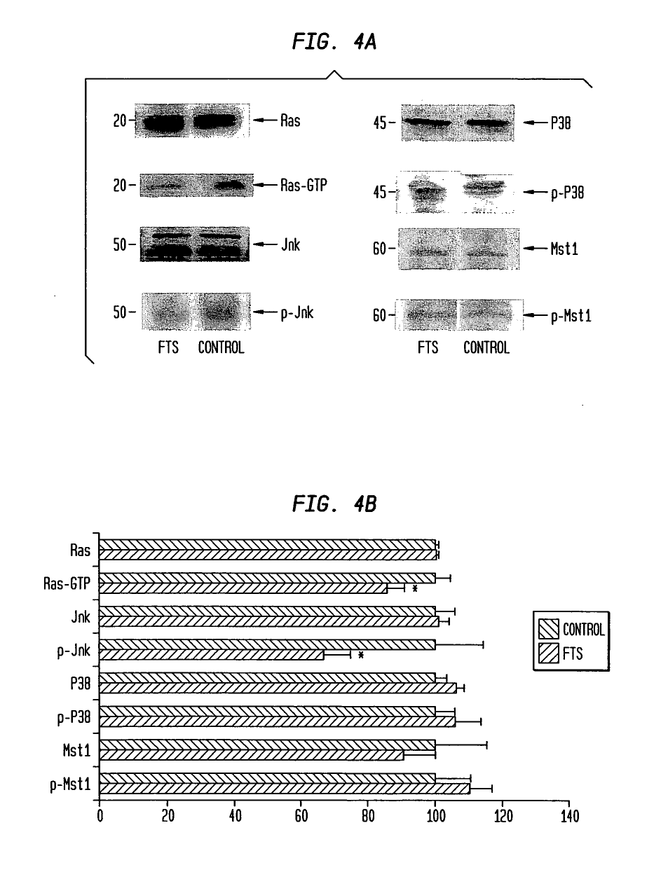

To examine the effect of FTS on Ras signaling on its prominent downstream effectors, Western immunoblotting using homogenates of the isolated perfused hearts was performed. Quantitative analysis of the immunoblots disclosed a reduction in levels of active Ras (Ras-GTP) and p-Jnk in hearts of the FTS group (p<0.05) at 30 minutes of reperfusion. No differences were observed in total Ras, Jnk, P38, p-P38, Mst1 and p-Mst1 levels between both groups (FIG. 4).

### V. FTS Administration Improved LV Function Post LAD Ligation In Vivo.

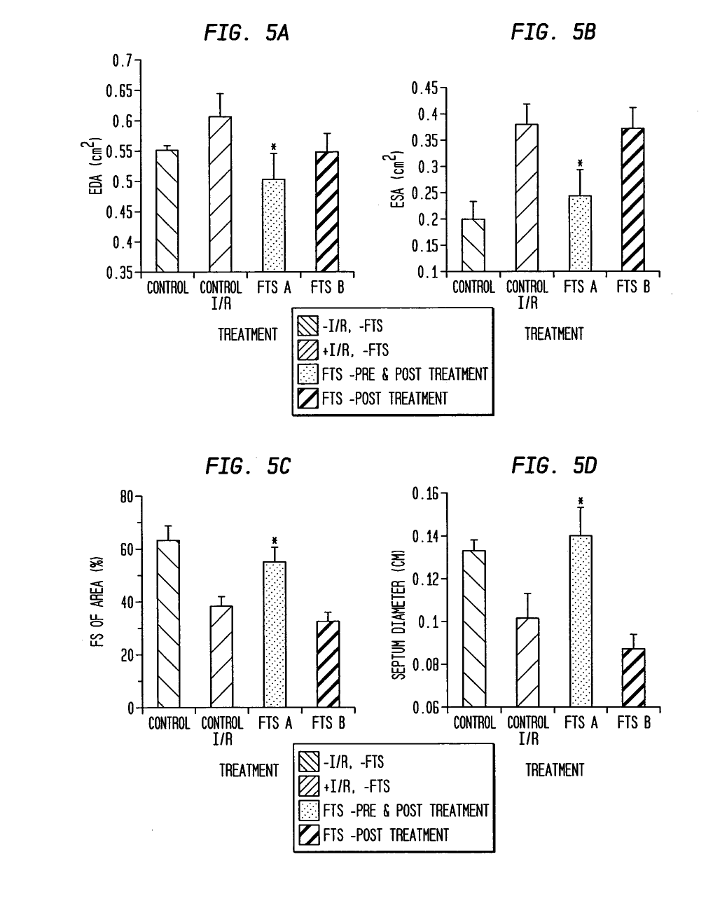

To examine the effect of systemic FTS treatment on the heart, in vivo experiments were conducted according two protocols. FTS administered 7 days prior to LAD ligation at a dose of 5 mg/kg and then for a period of 14 days (Protocol A) [prophylactic regimen] resulted in a significant improvement in most hemodynamic parameters of cardiac function compared with control I/R group. Left ventricular end-systolic and end-diastolic area (LVESA and LVEDA) were significantly better in the group administered FTS according to Protocol A compared with the control I/R group, p<0.05 (FIGS. 5A and 5B). The infarcted rats treated according to Protocol A also demonstrated significantly higher fractional shortening of area compared with the control I/R rats (55±6% vs. 38±4% respectively, p<0.05) (FIG. 5C). In addition, the septum diameter was significantly greater in the rats from the FTS group treated according to Protocol A as compared with the control I/R rats (0.14±0.01 vs. 0.1±0.01 respectively, p<0.05) (FIG. 5D).

When FTS (5 mg/kg) was administered according to Protocol B (14 days post-LAD ligation) [treatment regimen], no improvement in cardiac hemodynamic function was observed compared to control I/R (FIGS. 5A-5D).

### VI. FTS Attenuated I/R Injury In Vivo.

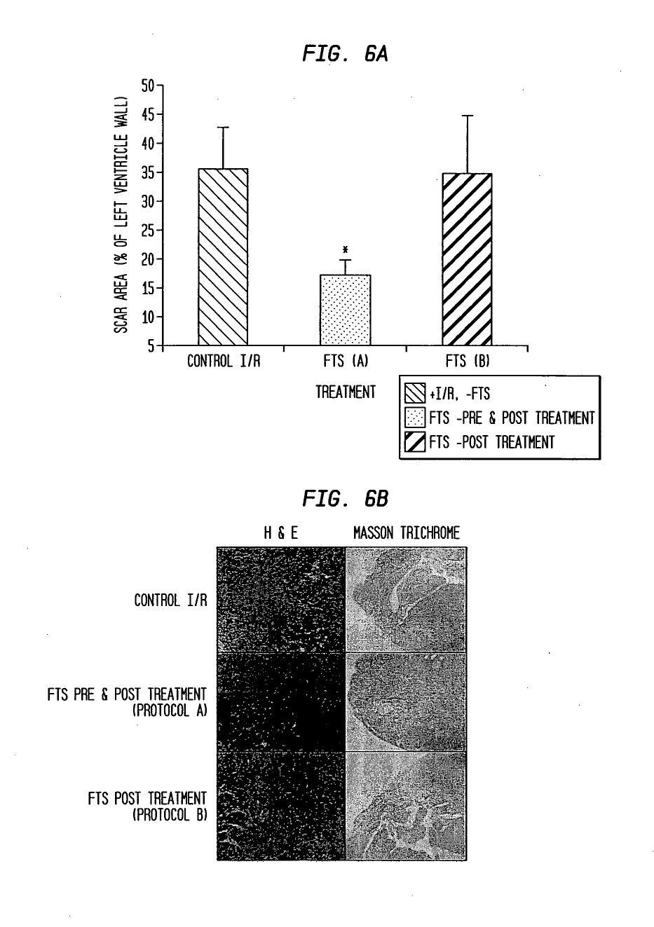

To determine the morphological damage caused to the heart tissue, heart sections were stained with H&E and Masson Trichrome. H&E staining reveals cardiomyocyte morphology and was used to evaluate the extent of damage caused to the cells. Masson Trichrome staining revealed collagen deposition in the heart tissue and allowed evaluation of scar size. Thus, both stains were used to evaluate the area of irreversible damage. Typical stained sections are shown in FIG. 6B. The area of irreversible damage is presented as a percentage of LV wall in FIG. 6A. Rats that received FTS according to Protocol A showed a significant reduction in the area of irreversible damage (17.25±2.5%) compared with control I/R rats (36±7%) and thus, improved cardiac function.

Rats that received FTS (5 mg/kg) according to Protocol B did not show a significant difference compared with control I/R group (FIG. 6A, p<0.05).

VII. FTS Downregulated Total Ras, Ras-GTP, p-Mrst1 In Vivo.

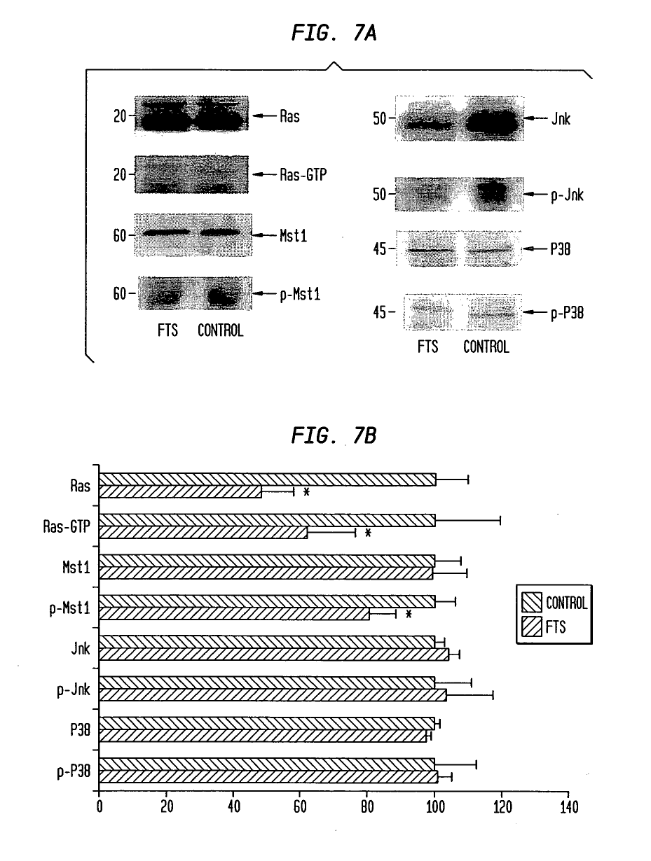

To determine the effects of FTS on Ras and its downstream effectors in vivo, Western immunoblotting was performed on the rats administered FTS according to Protocol A. Quantitative analysis of immunoblots demonstrated a reduction in levels of total Ras, active Ras (Ras-GTP), and p-Mst1 in hearts of rats from the FTS group (p<0.05). Levels of Mst, P38, p-P38, Jnk and p-Jnk did not differ in both groups (FIG. 7).

The publications cited in the specification, patent publications and non-patent publications, are indicative of the level of skill of those skilled in the art to which this invention pertains. All of these publications are herein incorporated by reference to the same extent as if each individual publication were specifically and individually indicated as being incorporated by reference.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1-12. (canceled)

13. A method of reducing the extent of myocardial ischemia/reperfusion injury, comprising administering to a human prior to reperfusion of the ischemic myocardium an effective amount of farnesylthiosalicylic acid (FTS) or an analog thereof as represented by the formula: 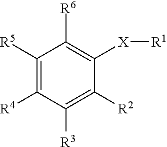
wherein
R¹ represents farnesyl or geranylgeranyl;
R² represents the groups COOR⁷, CONR⁷R⁸, wherein R⁷ and R⁸ are each independently hydrogen, alkyl or alkenyl, and COOM wherein M is a cation;
R³, R⁴, R⁵ and R⁶ are each independently hydrogen, alkyl, alkenyl, alkoxy, halo, trifluoromethyl, trifluoromethoxy, or alkylmercapto; and
X represents S; or a pharmaceutically acceptable salt thereof.

14. The method of claim 13, wherein the reperfusion injury of the ischemic myocardium results from a treatment selected from the group consisting of angioplasty, coronary artery bypass surgery, and thrombolytic therapy.

15. The method of claim 13, wherein the human is administered FTS.

16. The method of claim 13, wherein the human is administered an analog of FTS which is GGTS.

17. The method of claim 13, wherein FTS or its analog or a pharmaceutically acceptable salt thereof is administered orally.

18. The method of claim 13, wherein FTS or its analog or a pharmaceutically acceptable salt thereof is administered intravenously.

19. A method of treating myocardial ischemia/reperfusion injury, comprising administering to a human after reperfusion of the ischemic myocardium an effective amount of farnesylthiosalicylic acid (FTS) or an analog thereof as represented by the formula: 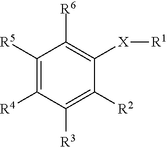
wherein
R¹ represents farnesyl or geranylgeranyl;
R² represents the groups COOR⁷, CONR⁷R⁸, wherein R⁷ and R⁸ are each independently hydrogen, alkyl or alkenyl, and COOM wherein M is a cation;
R³, R⁴, R⁵ and R⁶ are each independently hydrogen, alkyl, alkenyl, alkoxy, halo, trifluoromethyl, trifluoromethoxy, or alkylmercapto; and
X represents S; or a pharmaceutically acceptable salt thereof.

20. The method of claim 19, wherein the reperfusion injury of the ischemic myocardium results from a treatment selected from the group consisting of angioplasty, coronary artery bypass surgery, and thrombolytic therapy.

21. The method of claim 19, wherein the human is administered FTS.

22. The method of claim 19, wherein the human is administered an analog of FTS which is GGTS.

23. The method of claim 19, wherein FTS or its analog or a pharmaceutically acceptable salt thereof is administered orally.

24. The method of claim 19, wherein FTS or its analog or a pharmaceutically acceptable salt thereof is administered intravenously.

25-26. (canceled)

27. The method of claim 13, wherein the human is administered an analog of FTS which is 5-fluoro-FTS.

28. The method of claim 13, wherein the human is administered an analog of FTS which is FTS-methyl ester (FTSME).

29. The method of claim 13, wherein the human is administered an analog of FTS which is FTS-amide.

30. The method of claim 19, wherein the human is administered an analog of FTS which is 5-fluoro-FTS.

31. The method of claim 19, wherein the human is administered an analog of FTS which is FTS-methyl ester (FTSME).

32. The method of claim 19, wherein the human is administered an analog of FTS which is FTS-amide.
